(19) United States
(12) Patent Application Publication
(10) Pub. No.: US 2023/0355147 A1
(43) Pub. Date: Nov. 9, 2023

(54) ANALYTE SENSOR

(71) Applicant: PercuSense, Inc., VALENCIA, CA (US)

(72) Inventors: KAITLYNN OLCZAK, PORTER RANCH, CA (US); MIRELIS SANTOS CANCEL, SHERMAN OAKS, CA (US); RAJIV SHAH, RANCHO PALOS VERDES, CA (US); BRIAN KANNARD; BRADLEY LIANG, BLOOMFIELD HILLS, MI (US); ELIZABETH ESPINOZA, NORTHRIDGE, CA (US)

(73) Assignee: PercuSense, Inc., VALENCIA, CA (US)

(21) Appl. No.: 18/136,265

(22) Filed: Apr. 18, 2023

Related U.S. Application Data
(60) Provisional application No. 63/332,645, filed on Apr. 19, 2022.

(51) Int. Cl. A61B 5/1486

(52) U.S. Cl. CPC A61B 5/14865

(57) ABSTRACT

In one embodiment, a sensor to measure the presence of an analyte is disclosed. The sensor includes a working conductor with an electrode reactive surface. The sensor further includes a first reactive chemistry that is responsive to a first analyte and is in direct contact with the electrode reactive surface. The first reactive chemistry includes an enzyme, a first transport material, and an entrappable cofactor that includes a cofactor for the enzyme coupled to an anchor molecule. The sensor further includes a second transport material that enables diffusion of the first analyte to the first reactive chemistry.

### RELATED APPLICATION

This application claims the benefit of U.S. provisional application number 63/332,645 filed on Apr. 19, 2022. The application listed above is hereby incorporated by reference in its entirety for all purposes.

### FIELD OF THE INVENTION

The present invention is generally directed to devices and methods that perform in vivo monitoring of an analyte or analytes such as, but not limited glucose, lactate or ketones. In particular, the devices and methods are for electrochemical sensors that provide information regarding the presence or amount of an analyte or analytes within a subject.

### BACKGROUND OF THE INVENTION

Diabetes is a growing healthcare crisis, affecting nearly 30 million people in the United States. Approximately 10 percent of those affected require intensive glucose and insulin management. In hospital patients, hypoglycemia in both diabetic and non-diabetic patients is associated with increased cost and short- and long-term mortality.

Diabetic ketoacidosis (DKA) is a serious complication of diabetes. Diabetic ketoacidosis most often occurs in those with type 1 diabetes though it can also occur in those with other types of diabetes. DKA typically occurs when high levels of blood acids called ketones are produced. The condition develops is associated with diabetes because it is linked to the lack of insulin production. Without enough insulin, the body switches to burning fatty acids, which results in production of acidic ketone bodies.

Accordingly, it would be highly advantageous to enable real-time in-vivo detection and measurement of ketone bodies. The claimed invention seeks to address many issues associated with detecting and measuring ketone bodies or ketones.

### BRIEF SUMMARY OF THE INVENTION

In one embodiment, a sensor to measure the presence of an analyte is disclosed. The sensor includes a working conductor with an electrode reactive surface. The sensor further includes a first reactive chemistry that is responsive to a first analyte and is in direct contact with the electrode reactive surface. The first reactive chemistry includes an enzyme, a first transport material, and an entrappable cofactor that includes a cofactor for the enzyme being coupled to an anchor molecule. The sensor further includes a second transport material that enables diffusion of the first analyte to the first reactive chemistry.e.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1A includes exemplary views of a first side and a second side of a sensor, in accordance with embodiments of the present invention

FIG. 1B includes exemplary views of a first side and second side of another embodiment of the sensor, in accordance with embodiments of the present invention.

FIGS. 2A and 2B are exemplary illustrations of sensors that are configured with various combinations of single or multiple working electrodes, combined with single or multiple CR electrodes, or a separate counter electrode and reference electrode, in accordance with various embodiments of the present invention.

FIG. 3 is an illustration showing an exemplary process that enables or creates the entrappable cofactor that is mixed or blended or combined into the reactive chemistry, in accordance with embodiments of the present invention.

FIG. 4 is an exemplary illustration of the first reactive chemistry in accordance with embodiments of the present invention.

FIG. 5 is an exemplary illustration of optionally incorporating an electrically conductive element into the first reactive chemistry, in accordance with embodiments of the present invention.

FIGS. 6A and 6B are exemplary illustrations of alternative embodiments that incorporate additional materials, in accordance with embodiments of the present invention.

FIG. 7 is exemplary calibration data generated using a sensor configured as discussed above, in accordance with embodiments of the present invention.

### DETAILED DESCRIPTION

Presented below are embodiments of an electrode within a sensor that is intended to enable continuous real-time in-vivo electrochemical sensing of an analyte or molecule of interest within a subject. The in-vivo measurement within a subject is typically performed in tissue such as, but not limited to subcutaneous tissue. However, various embodiments can be inserted into the vasculature, musculature or organ tissue. The sensor may include a working electrode along with a counter electrode and a reference electrode. Alternatively, many embodiments utilize a working electrode in conjunction with a pseudo-reference electrode, alternatively referred to as a combined counter-reference electrode.

Embodiments of the sensor can be configured to measure analytes such as lactate, ketones, glucose and the like. Furthermore, while some embodiments may be configured to measure a single or individual analyte, other embodiments can be configured to measure multiple analytes including various combinations of at least two or more molecules of interest such as lactate, ketone, glucose, oxygen, reactive oxygen and the like. In still other embodiments, the sensors may be configured with infusion sets to enable sensing of a single or multiple molecule of interest while also enabling delivery of an infusate from a single point of entry.

In many embodiments electrochemical detection of the desired analyte is accomplished using an enzymatic reaction. In many embodiments presented below, an enzyme or enzymes are selected from the dehydrogenase family. Non-limiting, exemplary dehydrogenase enzymes include glucose dehydrogenase to measure glucose and 3-hydroxybutyrate dehydrogenase (3HBDH) to measure ketones. In other embodiments, especially those measuring multiple analytes, it may be possible to use alternate enzymes such as, oxidase based enzymes like glucose oxidase or lactate oxidase in addition to dehydrogenase base enzymes. In many embodiments, the enzyme is immobilized or trapped within a reactive chemistry. In some embodiments, the reactive chemistry includes a cofactor. The inclusion of the cofactor in sufficient quantity can improve the ability to measure analytes having generally low endogenous concentrations of cofactor by ensuring the kinetics of an enzyme and associated cofactor are limited by mass transfer of the molecule of interest. Particularly, inclusion of the cofactor can improve sensor properties such as, but not limited to, linearity of the sensor response to increasing concentrations of the analyte and increased duration of the sensor lifespan.

When dehydrogenase enzymes are used within the reactive chemistry, it may be beneficial to include or provide a cofactor. The inclusion of a cofactor within the sensor may be desirable because endogenous concentrations of the cofactor may be low or suboptimal for various design parameters of an electrochemical sensor. Accordingly, techniques or methods to incorporate or include a desired exogenous cofactor within the sensor may lead to improvements of in vivo sensor performance characteristics such as, but not limited to, sensitivity, linearity, and stability over time.

In many embodiments, it is desirable to locate an exogenous cofactor in close proximity to the corresponding enzyme. However, because the cofactor is often a much smaller molecule than the enzyme, it can be difficult to prevent the cofactor from migrating or moving away from the enzyme. To prevent migration or movement of the cofactor within the reactive chemistry, in many embodiments it is preferred to minimize diffusion, entrap or immobilize the cofactor in a location relatively close to the enzyme. Collocating sufficient amounts or quantities of the cofactor in close proximity to sufficient quantities of the enzyme enables the kinetics of the immobilized or entrapped enzyme and cofactor to be limited by mass transfer of the analyte or molecule of interest into the combined enzyme and cofactor structure. In some embodiments, retention or entrapment of the cofactor is accomplished by attaching the cofactor to an anchor molecule to create an entrappable cofactor. In these embodiments, the anchor molecule is a molecule having physical properties that minimize, reduce, or prevent migration or movement of the entrappable cofactor within the reactive chemistry that includes the enzyme. For example, an in-vivo sensor designed to continuously monitor or measure ketones, the reactive chemistry may utilize 3HBDH as the enzyme and may benefit from having exogenous cofactor NAD+ or NAD(P)+ entrapped, collocated, or immobilized in close proximity to the 3HBDH enzyme.

Disclosed below are exemplary, non-limiting embodiments of a sensor having a reactive chemistry that includes one or more enzymes along with at least a cofactor associated with at least one of the enzymes being collocated, entrapped, anchored or immobilized in close proximity to the enzyme. In many of the illustrations and much of the accompanying description, the exemplary non-limiting cofactor is NAD+ or NAD(P)+ because it is a cofactor commonly associated with the 3HBDH enzyme. However, other embodiments may use different cofactors that are either the natural cofactor, derivative of the natural cofactor, or a semi-synthetic or synthetic analogue of the natural cofactor that are compatible with the enzyme associated with detection or measurement of the molecule or analyte of interest. For example, in embodiments where the enzyme is 3HBDH, the natural cofactor NAD+. A derivative of the natural cofactor is NAD+ with a chemical modification or modifications, such as, but not limited to hexylamine NAD+. Additionally, a semi-synthetic or synthetic analogue of the natural cofactor that is enzymatically active. Additionally, the various embodiments discussed below should not be viewed as discrete embodiments. Rather, it is intended that various elements or components of the various embodiments are intended to be combined with elements, features or components of the other embodiments.

While embodiments and examples may be related to particular figures the scope of the disclosure and claims should not be construed to be limited to the explicit embodiments discussed. Rather it should be recognized that various combinations of features, elements and components can be interchanged, combined and even subtracted to enable other embodiments capable of continuous, real-time detection and measurement of an analyte or multiple analytes indicative of various metabolic conditions or general physiological health.

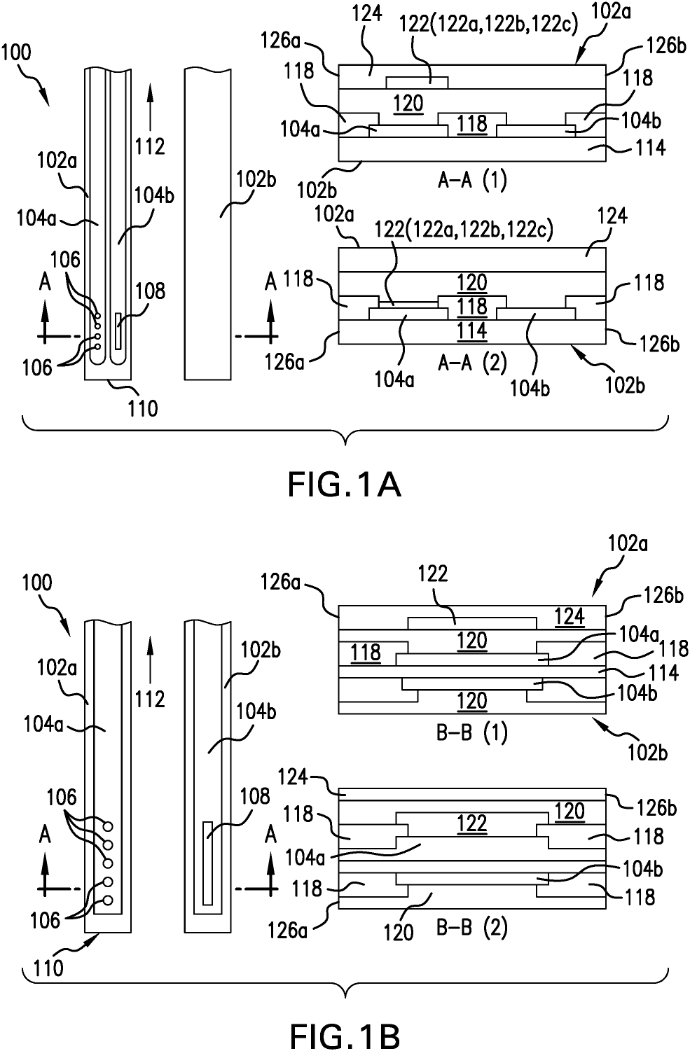

FIG. 1A includes exemplary views of a first side 102a and a second side 102b of a sensor 100, in accordance with embodiments of the present invention. FIG. 1A further includes exemplary cross-sections A-A-1 and A-A-2 of the sensor 100. As illustrated the sensor 100 includes at least two electrical traces. One of the electrical traces is configured to be a working trace 104a that supports working electrodes (WE) 106. A second electrical trace is configured to be a counter/reference (CR) trace 104b that supports a combined CR electrode (CRE) 108. Throughout this disclosure, the term working trace 104a may be interchanged with the term working conductor, working electrode conductor, or working electrode trace. Similarly, the term CR trace 104b may be interchanged with the term CR conductor, CR electrode trace, or CRE trace. The use of two electrical traces to support electrochemical sensing using a two electrode system should not be construed as limiting. In other embodiments, a three electrode system may be used where each of the working electrode, counter electrode and reference electrode are formed on their own electrical trace or conductor. With some embodiments it may be preferable to use two electrodes rather than three electrodes due to the decreased size of the sensor 100. However, in other embodiments it may be preferable to use a three electrode system in order to improve aspects of sensor performance such as sensitivity or linearity over time.

In FIG. 1A the WE 106 is illustrated as a plurality or array of apertures or openings, formed on the working trace 104a. The number of apertures or openings within the WE 106 array is intended to be exemplary and should not be construed as limiting. In various embodiments various configurations having more or fewer openings may be used to tune sensor performance based on the molecule being detected or sensed. Likewise, the illustration of the CRE 108 as a monolithic opening is intended to be illustrative rather than limiting. In other embodiments the CRE 108 may be formed from multiple openings on the CR trace 104b. Moreover, the ratio of area between the exposed WE 106 and CR electrode 108 provided in the illustration is intended to be exemplary rather than limiting.

In FIG. 1A both the WE 106 and the CRE 108 are exposed on the first side 102a. In preferred embodiments, the WE trace 104a and the CR electrode trace 104b are formed from a contiguous piece of electrical conductor, such as, but not limited to, stainless steel, silver, gold, platinum, or the like that is patterned to produce the working trace 104a and the CR trace 104b. In other embodiments other techniques such as deposition, machining, etching and the like can be used to form or place one or more of the working trace 104a and the CR electrode trace 104b. The WE 106 and CRE 108 are formed toward a distal end 110 of the sensor 100. Though not included in the illustration, it should be understood that the WE 104a and the CR electrode trace 104b include contact pads formed closer to a proximal end 112 that enable the sensor 100 to be connected to electronic components that power and operate the sensor 100.

Cross-sections A-A(1) and A-A(2) are exemplary illustrations of embodiments of the sensor 100, in accordance with various embodiments of the present invention. Common elements shared between the cross-sections A-A(1) and A-A(2) include an insulation 114 that is coupled to the WE trace 104a and the CR trace 104b. In embodiments where the WE 106 and CRE 108 are formed on the first side 102a, the second side 102b illustrated in FIG. 1A is the insulation 114. In many embodiments, the insulation 114 is an insulator such as, but not limited to a non-conductive film like polyimide. In other embodiments, different electrical insulators such as a solder mask may be used as the insulation 114. The specific embodiments discussed are intended to be exemplary and other non-conductive insulator materials may be used for insulation 114.

Insulation 118 covers working trace 104a and the CR trace 104b and the insulation has openings, windows, or apertures that expose a portion of the respective trace that enables formation of the WE 106 or the CRE 108. In many embodiments insulation 118 is selected from electrical insulators that can be applied over the respective electrode traces. Exemplary insulation 118 can include, but are not limited to non-conductive films like polyimide that are coupled to the electrode traces with adhesives. In other embodiments insulation 118 is selectively applied via spin coating, spraying, or alternate forms of depositing or placing an electrical insulator over the respective electrodes. In many embodiments, the opening or apertures within the insulation 118 that expose a portion of the underlying respective electrical trace are made using techniques such as, but not limited to, laser ablation, mechanical cutting, masking, etching or the like. The techniques to create the apertures are not intended to be limiting. Any technique that can be used to selectively apply or remove the insulation 118 from the respective electrical traces should be considered within the scope of this disclosure. For example, in some embodiments the insulation 118 may be printed over an electrical trace leaving the apertures exposed thereby obviating the need to remove the insulation to form the aperture. As illustrated the openings in the insulation 118 that expose the working conductor 104a are circular but that should not be construed as limiting. Rather, any shape opening or aperture within the insulation 118 should be considered within the scope of this disclosure. Similarly, the shape of the opening in the insulation 1180 that exposes the CR conductor 104b is intended to be exemplary and may be any variety of shape.

Additional common element shared between the cross-sections A-A (1) and A-A (2) is a first reactive chemistry 122 that in many embodiments further includes an enzyme 122a, a first transport material 122b and an entrappable cofactor 122c. Another common element between cross-sections A-A (1) and A-A (2) is a second transport material 120. In cross-section A-A (1) the second transport material 120 is applied over the insulation 118 and the working conductor 104a and the CR conductor 104b. In preferred embodiments the second transport material 120 is a hydrogel that extends from an edge 126a to an edge 126b. The purpose of the second transport material 120 is to enable unencumbered movement, diffusion or transport of molecules within fluid surrounding the sensor 100. Accordingly, after a hydration period, the second transport material 120 is intended to establish molecular concentrations within the second transport material 120 that are substantially identical to those within the fluid surrounding the sensor (other than those molecules that are intended to react within the sensor 100).

A first reactive chemistry 122 is applied over the second transport material 120 in substantial alignment with the exposed WE trace 104a to create a working electrode 106. The first reactive chemistry includes the enzyme 122a, the first transport material 122b and the entrappable cofactor 122c. In many embodiments, both the enzyme 122a and the entrappable cofactor are selected based on a molecule intended to be measured or detected by the working electrode 106. For example, in embodiments where ketones are the molecule of interest, the enzyme 122a included within the first reactive chemistry 122 may include a dehydrogenase based enzyme such as 3HBDH. The use of 3HBDH should not be construed as limiting as other embodiments may require the use of molecule specific enzymes. Non-limiting examples of other enzymes 122a include, but are not limited to detection or measuring of analytes or molecules of interest like glucose or lactate that are capable of being detected or measured with oxidase enzymes such as glucose or lactate oxidase or dehydrogenase based enzymes such as glucose or lactate dehydrogenase. In embodiments utilizing dehydrogenase based enzymes, it may be advantageous or necessary to include supplemental or additional cofactor to enable electrochemical sensing of the molecule of interest.

Accordingly, in many embodiments, the reactive chemistry 122 further includes an entrappable cofactor 122c along with a first transport material 122b. In many embodiments, the first transport material 122b is a polymer that is crosslinked. Crosslinking of the first transport material defines a porosity that entraps or ensnares the enzyme 122a and the entrappable cofactor 122c. In many embodiments, the first transport material 122b is selected from a family of hydrogels having similar properties to those of the second transport material. In preferred embodiments, when the reactive chemistry 122, a mixture or compound including the enzyme 122a, the first transport material 122b and the entrappable cofactor 122c is crosslinked or cured, the first transport material 122b creates a porous matrix that minimizes or prevents migration or movement of both the enzyme 122a and the entrappable cofactor 122c within or throughout the first transport materials 122b.

In preferred embodiments, the porosity of the first transport material 122b is selected to restrict or minimize migration or movement of the enzyme 122a and the entrappable cofactor 122c while allowing unrestricted or minimally restricted migration or movement of the analyte of interest, endogenous cofactor, and other endogenous molecules or compounds found within interstitial fluid within a subject. Retaining the entrappable cofactor 122c within the first reactive chemistry 122 supplements or enhances endogenous cofactor within a subject that may exist in generally low concentrations. Because endogenous cofactor in low concentrations may be quickly depleted within the sensor, the inclusion of the entrappable cofactor 122c is intended to improve or enhance continuous sensor performance over an expected sensor life that can be measured in multiple hours, days or weeks.

The porosity of the first transport material 122b is intended to freely enable diffusion or transport of endogenous cofactor which necessitates the purposeful modification or tuning of the entrappable cofactor 122c to achieve a desired diffusivity of the entrappable cofactor within the first transport material 122b. Specifically, the entrappable cofactor 122b is modified to minimize or prevent diffusion of the entrappable cofactor 122b within or throughout the first transport material 122b. With diffusion within the first transport material minimized, the entrappable cofactor 122b is retained in close proximity to the enzyme 122a while still enabling diffusion or transmission fluids containing endogenous cofactor, the analyte of interest and other molecules or compounds found in fluid surrounding the sensor within and throughout the first transport material 122b. The doping or addition of entrappable cofactor 122b that cannot migrate or diffuse away from the enzyme 122a improves sensor performance by compensating for any shortage of endogenous cofactor within a subject.

In many embodiments the shape of the first reactive chemistry 122 mirrors, or is at least similar to the shape of the aperture in the insulation 118. However, it should be noted that the first reactive chemistry 122 and a portion of the exposed WE trace may be the same shape or different shapes. Similarly, the exposed WE trace and the first reactive chemistry 122 may be nominally identical in size, or one may be larger or smaller than the other. In many embodiments, the first reactive chemistry 122 is sized and positioned such that if the first reactive chemistry 122 were projected upon the exposed working trace 104a, it would overlap or at least cast a shadow over at least some of the exposed working trace. The overlap or shadow cast by the first reactive chemistry 122 ensures that byproducts of the reaction between the molecule of interest and the first reactive chemistry 122 have a substantially direct path from the first reactive chemistry 122, through the second transport material 120, to the exposed working trace.

A third transport material 124 is optional and may be applied over both the first reactive chemistry 122 and the second transport material 120. Though illustrated as being applied from edge 126a to edge 126b, in various embodiments the third transport material 124 may be applied such that it does not extend to either, or both edges 126a and 126b. In some embodiments the third transport material 124 is impervious to the molecule of interest. In other embodiments, the third transport material 124 enables fluid surrounding an implanted sensor containing the analyte of interest, the cofactor and other molecules and compounds within the fluid to freely migrate or move throughout the third transport material 124. In many embodiments, the third transport material 124 is similar or identical to either or both of the first transport material and the second transport material.

Cross-section A-A (2) differs from cross-section A-A (1) because with A-A (2), the first reactive chemistry 122 is applied to the exposed WE trace 104a. In cross-section A-A (1), the first reactive chemistry 122 is not in direct contact with the WE trace 104a. Rather, with cross-section A-A (1), the first reactive chemistry is separated from the WE trace 104a by the second transport material 120. In cross-section A-A(1), an optional third transport material 124 may be applied over both the second transport material 120 and the first reactive chemistry 122. As illustrated in cross-section A-A (1), the first reactive chemistry 122 is sandwiched or encapsulated between the second transport material 120 and the third transport material 124. In many of these embodiments, the third transport material is intended to promote biocompatibility of the sensor by preventing or minimizing the likelihood that body fluid around an implanted sensor interacts directly with the first reactive chemistry 122. Additionally, in cross-section A-A (2), optional third transport material 124 is applied over the second transport material 120. In some embodiments where the second transport material 120 is applied directly over and in contact with the first reactive chemistry 122, the second transport material 120 operates as a biocompatibility layer. Specifically, in these embodiments the second transport material 120 operates as a barrier to minimize the likelihood of interstitial fluid surrounding an implanted sensor from directly interacting with the reactive chemistry 122.

FIG. 1B includes exemplary views of a first side 102a and second side 102b of another embodiment of the sensor 100, in accordance with embodiments of the present invention. FIG. 1B further includes exemplary cross-sections B-B (1) and B-B (2) of the sensor 100. As illustrated in FIG. 1B, the working trace 104a is formed on the first side 102a and the CR trace 104b is formed on the second side 102b. Similar to FIG. 1A, the use of a two electrode system is exemplary rather than limiting as other embodiments can use a three electrode system having separate traces for each of the working, counter and reference electrodes. In such embodiments, splitting the three electrodes across the first side 102a and second side 102b results in various permutations or combinations that should be considered within the scope of this disclosure despite the focus being on the exemplary two electrode system. It may be preferable to use an embodiment similar to that illustrated in FIG. 1B to reduce the overall width of the distal end 110, despite the increase in thickness of the edges 126a and 126b.

In FIG. 1B, the working electrode 106 is illustrated as a plurality of openings, or an array of openings or apertures, formed on the working trace 104a while the CR electrode 108 is illustrated as a single opening formed on the CR trace 104b. Similar to the embodiment illustrated in FIG. 1A, the CR electrode 108 is a single relatively large opening on the CR trace 104b that should not be construed as limiting. Rather the CR electrode 108 may also be configured as an array made up of multiple openings on the CR trace 104b. The exemplary number of openings within the WE 106 array should not be construed as limiting as other embodiments may include an WE 106 with an array with fewer or additional openings. Changing the number of openings may be used to tune sensor performance based on the relative concentration of the molecule being sensed. For example if there is a relatively low concentration of the molecule being sensed or detected, it may be advantageous to increase the number of openings within the WE 106 array. Alternatively, in embodiments where the concentration of the molecule being sensed is relatively high, decreasing the number of openings within the WE 106 array may result in satisfactory sensor performance while reducing manufacturing cost and complexity.

In addition to the number of openings within the array of working electrodes, sensor performance may be further tuned or enhanced by changing the surface area exposed within each of the openings via removal of more or less insulation 118. Larger openings may enable greater quantities of first reactive chemistry (e.g., enzyme) to be used thereby potentially generating higher electrical currents. Conversely, smaller openings may enable smaller quantities of a first reactive chemistry 122 (e.g., enzyme) to be used to generate sufficient electrical current to enable electrochemical sensing of the molecule of interest.

With the WE 106 being formed on the first side 102a and the CR electrode 108 being formed on the second side 102b, the respective electrical traces are electrically isolated from each other with insulation 114 (cross-sections B-B (1) and B-B (2)). In preferred embodiments, the working trace 104a and CR trace 104b are made from a flexible, electrically conductive material such as, but not limited to stainless steel. In alternative embodiments alternate materials other than stainless steel may be used to form one or more of the electrical traces. Non-limiting examples include, but are not limited to minimally or non-corrosive, electrically conductive and flexible materials such as gold, silver, platinum or alloys thereof. Still other materials include carbon nano-tubes or other conductive materials with a non-corrosive coating applied thereto. In preferred embodiments the WE trace is created by patterning a contiguous piece of the electrical conductor. Similarly, the CR trace may be created by patterning a separate piece of electrical conductor.

Cross-sections B-B (1) and B-B (2) are exemplary illustrations of embodiments of the sensor 100, in accordance with embodiments of the present invention. Similar to FIG. 1A, the cross-sections include insulation 114 and insulation 118 that are coupled to both working trace 104a and CR trace 104b. In some embodiments insulation 114 is a single layer of insulating material that is coupled to the working trace 104a and the CR trace 104b using adhesives. In other embodiments, insulation 114 is made of multiple non-conductive insulators laminated together that are coupled to the working trace 104a and the CR trace 104b. Non-limiting examples that can be used for insulation 114 and insulation 118 include polyamide, solder mask and similar materials. Non-limiting examples of the adhesives that can be used to couple the respective traces 104a and 104b to the insulation 114 and/or insulation 118 include acrylics or epoxies.

The WE 106 and CR electrode 108 are formed by creating apertures or opening within the insulation 118 that covers the respective electrical trace. The opening in the insulation 118 exposes a portion of either the WE trace 104a or the CR electrode trace 104b. The openings or apertures within the insulation 118 can be formed using laser ablation, mechanical cutting, masking and etching or any other technique to selectively remove a portion of the insulation 118 to expose the respective underlying electrical trace. Alternatively, in other embodiments rather than removing insulation 118 to expose an underlying electrical trace, the insulation 118 may be applied already having apertures formed in the insulation 118. As discussed regarding FIG. 1A, the shape of the apertures or openings in FIG. 1B are exemplary and should not be construed as limiting. Various embodiments may have apertures of various sizes, shapes and areas depending on the performance characteristics of the sensor 100.

Cross-sections B-B (1) and B-B(2) have the second transport material 120 applied over the insulation 118 and the exposed portion of the CR trace 104b. Cross-section B-B(1) also has the second transport material 120 applied over the insulation 118 and the exposed portion of the WE trace 104a. Cross-section B-B (1) has the first reactive chemistry 122 located over, but separated from the WE trace 104a, by the second transport material 120. Cross-section B-B(2) has the first reactive chemistry selectively applied over a portion of the insulation 118 and the WE trace 104a. It should be noted that the embodiments illustrated are exemplary and alternative embodiments that modify aspects of the exemplary embodiments should be considered within the scope of the disclosure. For example, embodiments similar to A-A(2) of FIGS. 1A and B-B(2) of FIG. 1B may partially fill the aperture or window of the insulation 118 thereby leaving a portion of the working conductor 104a exposed to be subsequently covered by first transport material 120. Similarly, in FIGS. A-A (1) and B-B (1) the reactive chemistry 122 overlap portions of the insulation 118 rather than being similar in size to the opening or aperture in the insulation 118.

In both FIGS. B-B (1) and B-B (2), a third transport material 124 may be optionally applied. In FIGS. B-B (1), the third transport material 124 is applied over both the first transport material 120 and the reactive chemistry 122. In FIGS. B-B (2), the third transport material 124 may be optionally applied over the second transport material 120. In various embodiments, the optional third transport material 124 may be applied across the entire sensor 100 from edge 126a to edge 126b. In other embodiments, the third transport material 124 does not extend from edge 126a to edge 126b.

In some embodiments the third transport material 124 is similar or identical to the second transport material 120. In other embodiments, the third transport material 124 is selected based on other properties such as, but not limited to, its ability to enable or prevent transmission of one or more molecules or compounds associated with the electrochemical reaction between the analyte of interest and the reactive chemistry.

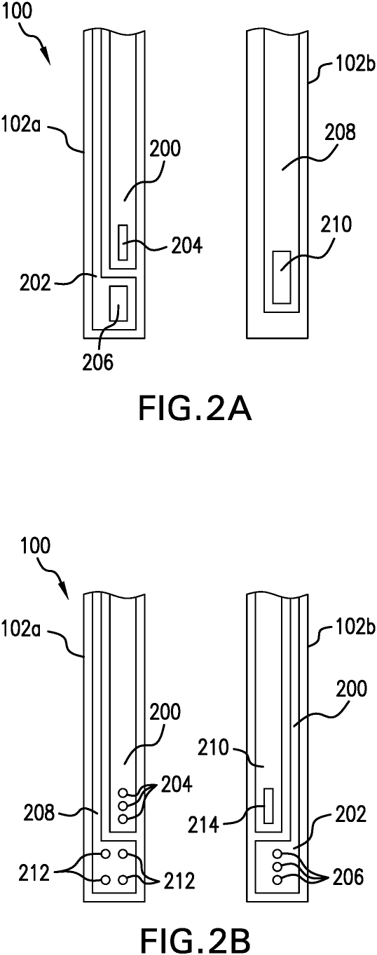

FIGS. 2A and 2B are exemplary illustrations of sensors 100 that are configured with various combinations of single or multiple working electrodes, combined with single or multiple CR electrodes, or a separate counter electrode and reference electrode, in accordance with various embodiments of the present invention. FIG. 2A is illustrative of a sensor 100 having a first trace 200 and a second trace 202 on the first side 102a a third trace 208 on the second side 102b. In one embodiment, the different traces can be configured to have two working electrodes and a combined counter/reference electrode that is shared between the two working electrodes. In other embodiments, the different traces can be configured to have a single working electrode, a discrete counter electrode, and a discrete reference electrode.

FIG. 2B is illustrative of a sensor 100 having a first trace 200 and a second trace 202 on the first side 102a. Additionally, in FIG. 2B, the sensor includes a third trace 208 and a fourth trace on the second side 102b. Accordingly, in some embodiments the first trace 200 may be configured to be a CRE and the second trace 202 is configured to be a first WE for a first molecule of interest. This leaves the third trace to be another CRE and the fourth trace to be a second WE for a second molecule of interest. Alternatively, two of the traces can be configured as a first and second WE while the third trace is configured as a discrete counter electrode and the fourth trace is configured as a discrete reference electrode. Such an embodiment would enable detection of two molecules of interest on different working electrodes both sharing a counter electrode and a working electrode.

In the embodiments discussed above, any of the reactive chemistries may incorporate or include various enzymes depending on the molecule or analyte of interest. Various types of enzymes that may be included as part of the reactive chemistry include, but are not limited to dehydrogenase based enzymes and/or oxidase based enzymes. In embodiments utilizing dehydrogenase based enzymes it may be desirable or preferable to incorporate a corresponding cofactor within close proximity to the enzyme. For example, if the analyte of interest is ketones, 3HBDH is the dehydrogenase based enzyme and inclusion or addition of the cofactor NAD+ or NAD(P)+ in close proximity to the 3HBDH may improve sensor performance. Accordingly, in many embodiments, it may be preferable to entrap or retain NAD+ within the reactive chemistry. Because of the relatively low molecular weight of a cofactor relative to both the enzyme and the molecule or analyte of interest, an alternative perspective may be to view the entrapment or retention of the cofactor as minimizing movement or at least the mobility or likelihood of movement of the cofactor within the reactive chemistry.

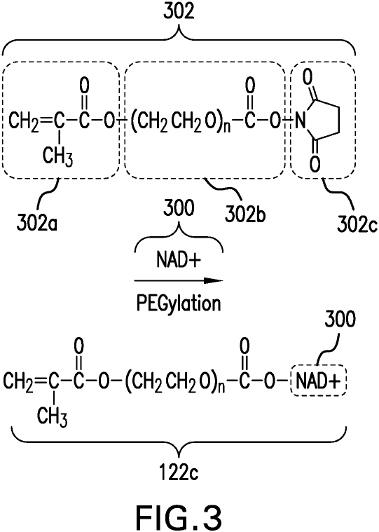

FIG. 3 is an illustration showing an exemplary process that enables or creates the entrappable cofactor 122c that is mixed or blended or combined into the reactive chemistry, in accordance with embodiments of the present invention. The process described below is intended to attach, couple or bond a desired cofactor 300 to an anchor molecule 302 to create the entrappable cofactor 122c that is a combination of a portion of the anchor molecule 302 and the cofactor 300. The intent of the process is to modify or tune the diffusivity of the resulting entrappable cofactor 122c to enable, improve or enhance its ability to be entrapped, immobilized or contained within the reactive chemistry. The exemplary process illustrated in FIG. 3 uses a methacrylate functional group 302a, a polyethylene glycol (PEG) backbone 302b and succinimide group 302c as the anchor molecule 302. The process replaces the succinimide group 302c of the anchor molecule 302 with the desired cofactor 300 resulting in the entrappable cofactor 122c.

The use of a PEG backbone as the anchor molecule 302 for the entrappable cofactor 122c is intended to be exemplary rather than limiting. In other embodiments other anchor molecules 302 such as, but not limited to polymers like polyethyleneimine and alginate may be used to tune the diffusivity of the entrappable cofactor within the first transport material. In some embodiments tuning or modification of the diffusivity is accomplished by attaching the cofactor 300 to an anchor molecule 302 that significantly increases the molecular size and/or molecular weight of the resulting entrappable cofactor 122c. While a preferred molecular mass may be dependent upon multiple variables such as, but not limited to, porosity of the first transport material and molecular mass of the cofactor 300, in many embodiments and exemplary range for the molecular mass of the anchor molecule 302 is between 100 and 100,000 daltons. In still other embodiments, another exemplary range for the molecular mass of the anchor molecule 302 is between 500 and 50,000 daltons. In many preferred embodiments, the molecular mass of the anchor molecule 302 falls between a range of 1,000 and 20,000 daltons. Accordingly, the entrappable cofactor 122c that includes the cofactor 300 and anchor molecule 302 of significant size or preferred molecular mass has a decreased diffusivity (alternatively, increased entrapability) compared to just the cofactor 300 within the porous matrix of the first transport material.

In alternative embodiments, rather than relying on molecular mass or physical size relative to porosity of the first transport material, the cofactor 300 can be attached to an anchor molecule that imparts an electrical charge to the resulting entrappable cofactor 122c that promotes entrapment, immobilization or retention of the entrappable cofactor 122c within the reactive chemistry. In embodiments where the electrical charge associated with the anchor molecule 302 reduces diffusivity, electrical charge of the first transport material may be more relevant than porosity. Alternatively, in embodiments having electrically charged anchor molecules 302, the first reactive chemistry may further include a specific molecule having an electrical charge that is opposite to the charge of the anchor molecule 302. In still other embodiments, an anchor molecule 302 can be selected based on size and/or molecular mass and its ability to impart an electrical charge. In these embodiments, the remainder of elements within the first reactive chemistry (e.g., the first transport material, the enzyme or even supplemental compounds or molecules) may be selected based on both porosity and an electrical charge that attracts the anchor molecule)

The exemplary PEGylation process illustrated in FIG. 3 is used to replace a succinimide group 302c with a specific or desired cofactor 300. In FIG. 3, the desired cofactor 300 is NAD+ or NAD(P)+ but that should not be construed as limiting. Throughout this disclosure it should be understood that the cofactor may be selected based on a single or multiple requirements or preferences such as, but not limited to, an enzyme or enzymes being used, an analyte or analytes being detected or sensed, and the general availability of endogenous cofactor associated with either an analyte, an enzyme, or analytes or enzymes. Additionally, the inclusion of the succinimide group 302c on the PEG backbone 302b should be viewed as illustrative rather than limiting. In other embodiments, the PEG backbone 302b can include functional groups other than, or in addition to the succinimide group illustrated. Additional exemplary function groups include, but are not limited to, carboxylic acid, amine and biotin. Moreover, in still other embodiments, rather than replacing a single functional group, an anchor molecule may have multiple attachment locations from one or more functional groups that can be replaced with cofactor, other molecules with desired properties, or other components of the reactive chemistry or sensor.

As discussed above, selection of the anchor molecule can be influenced by the desired molecular size and/or molecular weight of the resulting entrappable cofactor 122c (i.e., the anchor molecule that includes the methacrylate functional group, the PEG backbone, and the desired cofactor). Selection of the molecular size or weight of the PEGylated cofactor can be used to improve entrapability of the cofactor within the first transport material incorporated into the reactive chemistry thereby improving overall sensor stability and/or functionality of the sensor.

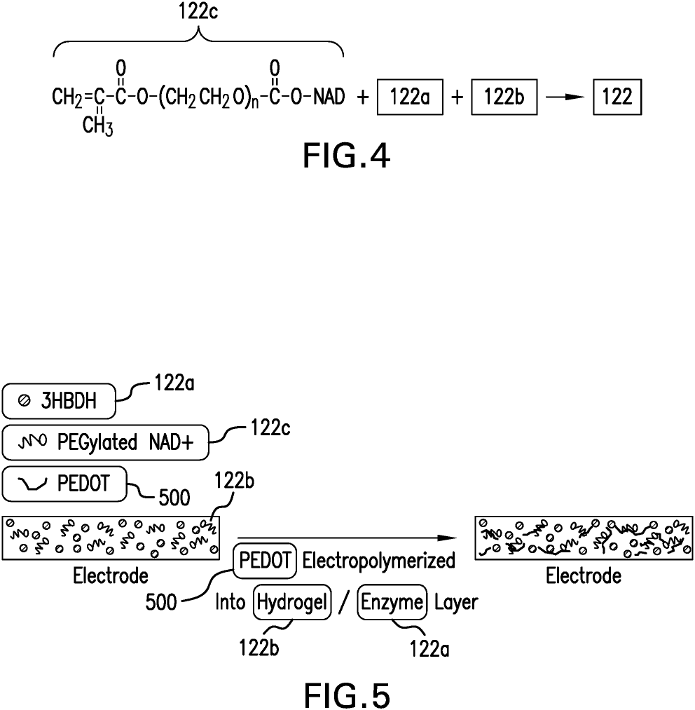

FIG. 4 is an exemplary illustration of the first reactive chemistry 122 in accordance with embodiments of the present invention. In FIG. 4, the entrappable cofactor 122c created during the process described above is combined, mixed, or blended with an enzyme 122a and the first transport material 122b to create a first reactive chemistry 122. Closely collocating sufficient entrappable cofactor 122c in close proximity to the enzyme 122a within the first transport material 122b to create the first reactive chemistry structure enables kinetics of reactions between the enzyme and cofactor to be limited by mass transfer of the molecule of interest into the first reactive chemistry structure. In FIG. 4, the enzyme 122a is specified broadly as being a dehydrogenase enzyme. However, in different embodiments alternative types of enzymes may be used depending on the desired reaction and analyte or molecule of interest. For example, in FIG. 4, the dehydrogenase enzyme 3HBDH would function with the entrappable cofactor 122c including either NAD+ or NAD(P)+.

Variables that can be controlled or tuned during the creation of the first reactive chemistry 122 include, but are not limited to the concentration or amount of the entrappable cofactor 122c, the amount of enzyme 122a or enzyme loading or enzyme concentration, and the amount of first transport material 122b. While there may be a preferred enzyme based on the entrappable cofactor (e.g., where the enzyme is 3HBDH the cofactor is NAD+ or NAD(P)+), in some embodiments additional enzymes may be mixed with the entrappable cofactor resulting in a reactive chemistry that includes multiple enzymes. Examples of multi-enzyme reactive chemistries include, but are not limited to multiple dehydrogenase enzymes or reactive chemistries that include various combinations of dehydrogenase enzymes and oxidase enzymes (e.g., 3HBDH and NADH-oxidase). In embodiments configured to measure ketones, exemplary concentrations of an enzyme like 3HBDH can vary between 1 -80 wt% with the weight percent being highly dependent on enzyme activity. In these embodiments, the exemplary concentration of entrappable cofactor NAD+ can vary between 5 -95 wt%, where the weight percent may be mostly determined by the size or weight of the selected anchor molecule. In many of these embodiments, the first transport material can vary between 1 -90 wt%, again, the weight percent being determined by the other constituents within the reactive chemistry and the desired physical properties of the reactive chemistry. It should be understood that the ranges provided are intended to be illustrative of embodiments based on exemplary design parameters. Accordingly, the examples discussed above should be construed as exemplary rather than limiting.

In addition to varying which enzyme or enzymes along with the respective concentrations of the respective enzymes and entrappable cofactor, selection of the first transport material may also be based on one or more of a desired degree of crosslinking, a desired cure time and a desired cure duration. Changing the degree of crosslinking via the first transport material can increase or decrease the mobility of the entrappable cofactor throughout the reactive chemistry. For example, assuming the exemplary entrappable cofactor remains the same, in embodiments with less crosslinking the entrappable cofactor may be able to migrate or diffuse more easily through the reactive chemistry compared to an embodiment where the first transport material has a higher degree of crosslinking.

An additional consideration when selecting the first transport material may also be the wavelength of light necessary to cure the first transport material being introduced into the first reactive chemistry. Still another variable associated with cure time and/or the cure wavelength includes the properties of the methacrylate functional group. In different embodiments, the selection of the methacrylate group in combination with the properties of the first transport material can be selected, tuned or optimized to achieve a desired polymerization of the first reactive chemistry. In many embodiments where the first transport material is a hydrogel, the hydrogel may include a photoinitiator to promote curing or crosslinking. In various embodiments, a separate or additional photoinitiator may be used to further refine or tune aspects associated with curing the first transport material.

FIG. 5 is an exemplary illustration of optionally incorporating an electrically conductive element 500 into the first reactive chemistry 122, in accordance with embodiments of the present invention. In FIG. 5, the left side is an exemplary illustration of the first reactive chemistry 122 described above. Specifically, the first reactive chemistry 122 includes the first transport material 122b, the enzyme 122a (3HBDH) and the entrappable cofactor 122c. As illustrated on the right side of FIG. 5, the addition, incorporation, or electropolymerization of the electrically conductive element 500, or conductive element, enables electrical contact between the electrode surface and the first reactive chemistry 122. Moreover, thoroughly mixing, incorporating, or electropolymerizing the conductive element 500 into or through the first reactive chemistry 122 enhances or improves charge transport throughout the first reactive chemistry 122. Thus, when the first reactive chemistry 122 that includes a conductive element 500 is in physical contact with the WE the conductive element 500 enables charge transport from the WE throughout the first reactive chemistry 122 including to both the enzyme 122a and the entrappable cofactor 122c.

In still other embodiments, to enable, enhance or improve electrical conductivity, the conductive element 500 may be incorporated into the various other transport materials associated with the sensor. For example, the conductive elements 500 may be optionally included in the second transport material to enable electrical conductivity through the second transport material, in embodiments where the first reactive chemistry is separated from the WE by the second transport material.

An exemplary, non-limiting conductive element includes various embodiments of conductive polymer such as poly(3,4-ethylenedioxythiophene)-poly(styrenesulfonate) commonly referred to as PEDOT-PSS. Conductive polymers like PEDOT-PSS enable electron transport via movement of delocalized electrons along the conjugated backbone. The conjugated backbone has alternating single and double bonds that result in overlapping p-orbitals that create an extended π-conjugated system. Interpolymer change transport is thus enabled through movement of delocalized electron movement through the conjugated system or electron transfer. It should be noted that electron transport using conductive polymers is different from electron transport using redox polymers. Specifically, electron transport with redox polymers occurs via electron transfer between spatially and electrostatically localized redox sites linked to a polymeric backbone.

Additional benefits of conductive polymers include the ability to modify the chemical structure to change the physical properties that in turn can change the interactions of the conductive polymer with the surrounding environment. For example, in some embodiments modifications to the chemical structure of the conductive polymer enables tuning of the hydrophobicity of the conductive polymer. Still other modifications to the conductive polymer enable tailoring of adhesion properties to a conducting or non-conducting material. In other embodiments, the conductive polymer may be modified in order to selectively functionalize portions or the entirety of the conductive polymer.

Another benefit of incorporating a conductive polymer within the first reactive chemistry are the multiple methods for polymerizing conductive polymers from their respective monomers. For example, in some embodiments the conductive polymer may be polymerized via chemical polymerization where preferred chemicals act as an oxidant. Non-limiting, exemplary chemicals to enable chemical polymerization include, but are not limited to metal ions, iron (III)-sulfonates, and the like. In other embodiments, the conductive polymer relies on electrochemical polymerization. Exemplary types of electrochemical polymerization includes, but are not limited to potentiostatic electrochemical polymerization and galvanostatic electrochemical polymerization.

Furthermore, with conductive polymers, the physical and electrochemical properties of the conductive polymer can be tuned by modifying preparation parameters such as, but not limited to the polymerization method, dopant selection and the like. In many embodiments, alternative conductive elements may be used such as, but not limited to carbon or graphene. In still other embodiments, the conductive element may be a combination of multiple conductive materials or elements such as conductive polymers and carbon nanotubes.

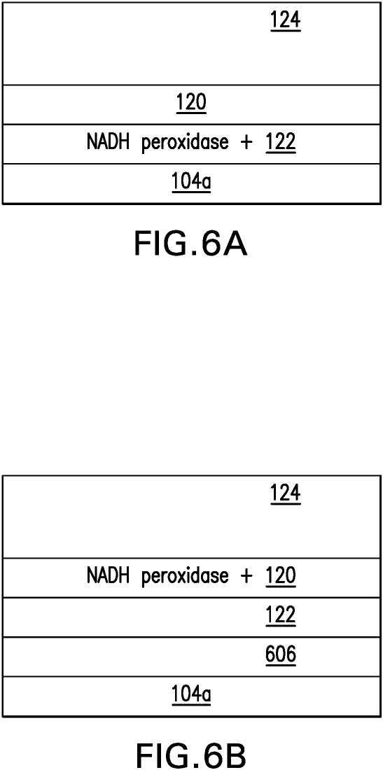

FIGS. 6A and 6B are exemplary illustrations of alternative embodiments that incorporate additional materials, in accordance with embodiments of the present invention. In FIGS. 6A and 6B additional or supplemental materials or compounds may be included or mixed into the first reactive chemistry 122 or alternatively, into the second transport material 120. An exemplary, non-limiting inclusion that may be optionally incorporated into either the first reactive chemistry 122 or the second transport material 120 is NADH peroxidase. In embodiments utilizing 3HBDH as the enzyme, the inclusion of NADH peroxidase can promote conversion of peroxide and NADH from fluid surrounding an implanted sensor into NAD+.

FIG. 6B further includes an optional surface preparation 600 that is located on at least a portion of the exposed working trace. In some embodiments the surface preparation 600 is the application of a mediator such as, but not limited to, poly-TBO. In other embodiments the surface preparation 600 includes the application of a surface enhancement that increases the surface area of the electrode surface.

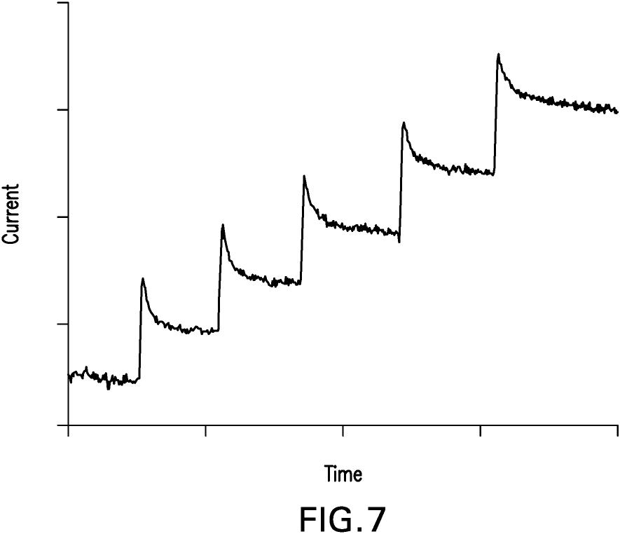

FIG. 7 is exemplary calibration data generated using a sensor configured as discussed above, in accordance with embodiments of the present invention. The data is a plot of electrical current versus time acquired from a sensor where the analyte of interest or molecule being detected was ketones. The sensor that generated the data used 3HBDH as the enzyme, a hydrogel as the first transport material and NAD+ attached to a PEG backbone as the entrappable cofactor.

The embodiments discussed above are intended to disclose the inclusion of an exogenous cofactor with tunable or controlled diffusion properties within an implantable in-vivo sensor to enable detection of sensing of an analyte or molecule of interest. The inclusion of the entrappable cofactor is intended to improve aspects of sensor performance such as sensitivity and duration of sensor life span by ensuring there is sufficient cofactor to react with the analyte of interest and enzyme to produce an electrochemical signal. Entrapping or minimizing diffusion of the cofactor within the reactive chemistry retains the cofactor in close proximity to the enzyme while also allowing endogenous cofactor, the molecule of interest and by-products of the electrochemical reaction to diffuse throughout the sensor.

As described above, modifying diffusivity of a cofactor for enzymatic electrochemical sensing may be accomplished using a variety of techniques such as modifying the cofactor to be an entrappable cofactor. An entrappable cofactor has properties that encumbers, retards or minimizes diffusion of the entrappable cofactor within a matrix or mixture of materials surrounding the cofactor. One technique to accomplish the reduced diffusivity of an entrappable cofactor is by manipulating or tuning the properties of both the entrappable cofactor and the material being mixed or blended with the entrappable cofactor.

Creating the entrappable cofactor can include modifying a cofactor to include an anchor molecule. The anchor molecule augmenting the properties of the cofactor to create an entrappable cofactor with preferred properties such as, but not limited to, molecular size, molecular weight or electrical charge. The exemplary embodiments discussed above should not be construed as limiting. In other embodiments an entrappable cofactor can achieve a desired size, weight or electrical charge by attaching or coupling the cofactor to various combinations of anchor molecule, enzyme, additional cofactor, or various combinations thereof. Thus, while FIG. 3 illustrates a cofactor attached to an anchor molecule, in various other embodiments, a cofactor may be bonded to an anchor molecule and enzyme may also be bonded to the same anchor molecule to create the entrappable cofactor.

In many embodiments, additional features or elements can be included, added or substituted for some or all of the exemplary features described above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. A sensor to measure the presence of an analyte, comprising:
a working conductor having an electrode reactive surface;
a first reactive chemistry being responsive to a first analyte, and in direct contact with the electrode reactive surface, the first reactive chemistry including an enzyme, a first transport material, and an entrappable cofactor that includes a cofactor for the enzyme coupled to an anchor molecule; and
a second transport material that enables flux of the first analyte to the first reactive chemistry.

2. The sensor to measure the presence of an analyte as described in claim 1, wherein the first transport material is a crosslinked polymer having a porosity based on a level of crosslinking.

3. The sensor to measure the presence of an analyte as described in claim 2, wherein the anchor molecule and the porosity define a preferred diffusivity of the entrappable cofactor within the first transport material.

4. The sensor to measure the presence of an analyte as described in claim 3, wherein the tunable diffusivity of the entrapable cofactor is selected to minimize diffusion of the entrappable cofactor throughout the first transport material.

5. The sensor to measure the presence of an analyte as described in claim 4 further including: an insulation layer covering the working conductor, the insulation layer having at least one aperture that exposes the electrode reactive surface.

6. The sensor to measure the presence of an analyte as described in claim 5, wherein the reactive chemistry is disposed within the aperture within the insulation layer.

7. The sensor to measure the presence of an analyte as described in claim 6, wherein the reactive chemistry covers a portion of the electrode reactive surface.

8. The sensor to measure the presence of an analyte as described in claim 6, wherein the reactive chemistry covers an entirety of the electrode reactive surface.

9. The sensor to measure the presence of an analyte as described in claim 8, wherein the reactive chemistry fills the aperture.

10. The sensor to measure the presence of an analyte as described in claim 9, wherein the reactive chemistry further covers a portion of the insulation.

11. The sensor to measure the presence of an analyte as described in claim 6, wherein the second transport material is applied over the insulation layer and the reactive chemistry.

12. The sensor to measure the presence of an analyte as described in claim 11, wherein the tunable diffusivity of the entrappable cofactor and the porosity of the first transport material are selected to minimize diffusion of the entrappable cofactor into the second transport material.

13. The sensor to measure the presence of an analyte as described in claim 6, wherein the reactive chemistry further includes a conductive polymer.

14. The sensor to measure the presence of an analyte as described in claim 1, wherein the anchor molecule has a molecular mass between 500 and 50,000 daltons.

15. The sensor to measure the presence of an analyte as described in claim 1, wherein a preferred electrical charge associated with the anchor molecule establishes the tunable diffusivity of the entrappable cofactor.

16. The sensor to measure the presence of an analyte as described in claim 1, wherein the anchor molecule has a molecular mass between 500 and 50,000 daltons and imparts a preferred electrical charge to the entrappable cofactor.

17. The sensor to measure the presence of an analyte as described in claim 15, wherein the first transport material has an electrical charge being opposite the preferred electrical charge of the anchor molecule.

18. The sensor to measure the presence of an analyte as described in claim 16, wherein the first transport material has a porosity that encumbers diffusion of molecules having a molecular mass greater than 500 daltons and an electrical change being opposite the preferred electrical charge of the anchor molecule.

19. The sensor to measure the presence of an analyte as described in claim 9, wherein the second transport material is applied over the reactive chemistry and a portion of the insulation.

20. The sensor to measure the presence of an analyte as described in claim 9, wherein the second transport material covers an entirety of the insulation layer and the reactive chemistry.
